(19) United States
(12) Patent Application Publication
(10) Pub. No.: US 2026/0308111 A1
(43) Pub. Date: Oct. 8, 2026

(54) SOLID STATE SALIFICATION OF ORGANIC MOLECULES

(71) Applicant: Florida State University Research Foundation, Inc., Tallahassee, FL (US)

(72) Inventors: Zachary T. Dowdell, Tallahassee, FL (US); Robert W. Schurko, Tallahassee, FL (US)

(21) Appl. No.: 19/637,394

(22) Filed: Apr. 2, 2026

Related U.S. Application Data
(60) Provisional application No. 63/782,712, filed on Apr. 3, 2025.

(51) Int. Cl. C07C 227/18

(52) U.S. Cl. CPC C07C 227/18

(57) ABSTRACT

In accordance with the purpose(s) of the present disclosure, as embodied and broadly described herein, the disclosure, in one aspect, relates to methods for producing a salt of a solid organic compound. The method involves (a) combining in the solid state the solid organic compound comprising one or more protonatable groups and an acid composition, wherein the amount of acid in the acid composition is about stoichiometric or less than stoichiometric relative to the one or more protonatable groups of the solid organic compound to produce a precursor mixture; and mixing the precursor mixture by mechanochemical milling to produce the salt of the solid organic compound. The methods described herein can produce salts of a variety of different organic molecules and is capable of being scaled up.

### CROSS-REFERENCE TO RELATED APPLICATIONS

This application claims the benefit of and priority to co-pending U.S. Provisional Patent Application No. 63/782,712, filed on Apr. 3, 2025, the contents of which are incorporated by reference herein in their entireties.

### STATEMENT ON FUNDING PROVIDED BY THE U.S. GOVERNMENT

This invention was made with Government support under contract DMR212556 awarded by the National Science Foundation. The Government has certain rights in the invention.

### BACKGROUND

Salts of organic molecules have widespread applications in several industries including pharmaceuticals, nutraceuticals, agrochemicals, and explosives. Typically, the free base of the organic compound is mixed with an excess amount of acid in a solvent to produce the corresponding salt. However, undesirable reactions such as acid hydrolysis can occur. Thus, it would be desirable to have alternative approaches to producing salts of organic compounds in a straight forward and efficient manner.

### SUMMARY

In accordance with the purpose(s) of the present disclosure, as embodied and broadly described herein, the disclosure, in one aspect, relates to methods for producing a salt of a solid organic compound. The method involves (a) combining in the solid state the solid organic compound comprising one or more protonatable groups and an acid composition, wherein the amount of acid in the acid composition is about stoichiometric or less than stoichiometric relative to the one or more protonatable groups of the solid organic compound to produce a precursor mixture; and mixing the precursor mixture by mechanochemical milling to produce the salt of the solid organic compound. The methods use acid compositions that have very little solvent (e.g., microliter scale relative to the amount of the organic molecule). The methods described herein can produce salts of a variety of different organic molecules and is capable of being scaled up.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows the synthetic pathways for the various solid forms of Picolinic Acid.

FIG. 2 shows the experimental and simulated PXRD patterns of nicotinamide and HCl salts of nicotinamide.

FIG. 3 shows the experimental and simulated PXRD patterns of nicotinamide and salts of nicotinamide.

FIG. 4 shows the experimental and simulated PXRD patterns of isonicotinamide and salts of isonicotinamide.

FIG. 5 shows the experimental and simulated PXRD patterns of picolinamide and salts of picolinamide.

FIG. 6 shows the experimental and simulated PXRD patterns of nicotinic acid and salts of nicotinic acid.

FIG. 7 shows the experimental and simulated PXRD patterns of isonicotinic acid and salts of isonicotinic acid.

FIG. 8 shows the experimental and simulated PXRD patterns of picolinic acid and HCl salts of picolinic acid.

FIG. 9 shows the experimental and simulated PXRD patterns of caffeine and HCl salts of caffeine.

FIG. 10 shows the experimental and simulated PXRD patterns of theophylline, theobromine, and respective HCl salts.

FIG. 11 shows the experimental and simulated PXRD patterns of acetaminophen and HCl salts of acetaminophen.

FIG. 12 shows the experimental and simulated PXRD patterns of metformin, creatine, and respective HCl salts.

FIG. 13 shows the experimental and simulated PXRD patterns of L-Lysine and respective polyheterosalts of L-Lysine.

FIG. 14 shows the experimental and simulated PXRD patterns of L-Histidine and salts of L-Histidine.

FIG. 15 shows the experimental and simulated PXRD patterns of L-Alanine and L-Alanine HCl.

FIG. 16 shows the experimental and simulated PXRD patterns of sulfanilamide HCl.

FIG. 17 shows the experimental and simulated PXRD patterns of isoniazid and salts of isoniazid.

FIG. 18 shows the experimental and simulated PXRD patterns of amoxicillin trihydrate and amoxicillin trihydrate milled with 1 and 3 equivalents of HCl.

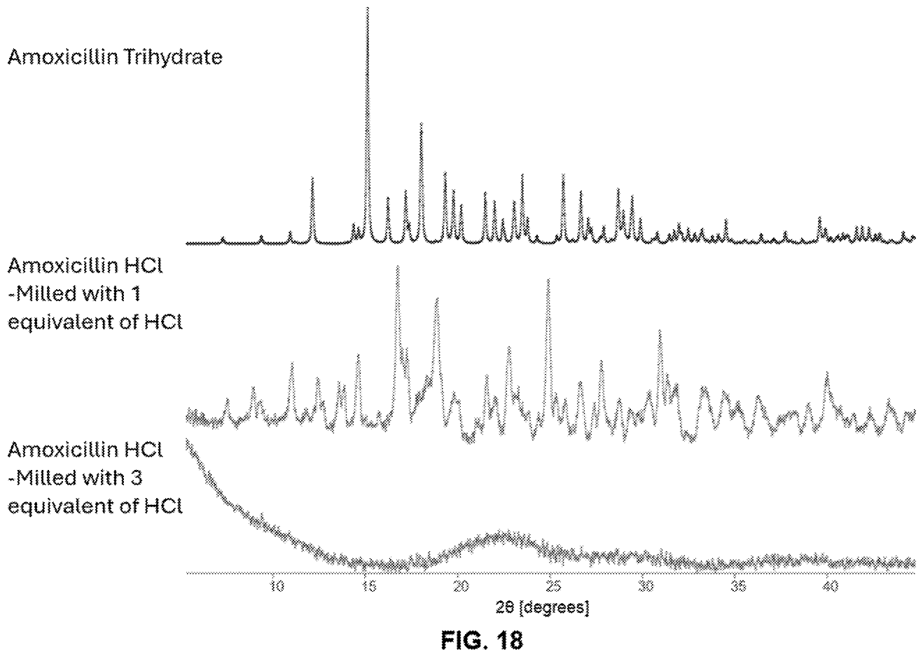

FIG. 19 shows ³⁵Cl static SSNMR spectra of NicH, NicH-Nic:H₂O, and NicAH acquired at 18.8 T.

FIG. 20 shows ³⁵Cl static SSNMR spectra of IsoH, IsoH-Iso:H₂O, and IsoAH acquired at 18.8 T

FIG. 21 shows ³⁵Cl static SSNMR of CaffH:H₂O and CaffH acquired at 18.8 T.

FIG. 22 shows the experimental and calculated PXRD patterns of Glu, GluHCl, GluHCl_{0.75}HBr_{0.25}, GluHCl_{0.50}HBr_{0.50}, GluHCl_{0.25}HBr_{0.75}, and GluHBr. The dashed yellow lines indicate key diffraction peaks that shift to the left as the concentration of Cl⁻ decreases. The dashed black line indicates a key diffraction peak in glutamic acid that is not present in any synthesized materials.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an excipient” include, but are not limited to, mixtures or combinations of two or more such excipients, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range. Thus, for example, if a component is in an amount of about 1%, 2%, 3%, 4%, or 5%, where any value can be a lower and upper endpoint of a range, then any range is contemplated between 1% and 5% (e.g., 1% to 3%, 2% to 4%, etc.).

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and Care disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance occurs and instances where it does not.

The term “a protonatable group” is a Lewis base that includes at least one pair of electrons capable of reacting with an acid such that the Leis base is protonated.

The term “a nitrogen bearing group” is a group that includes a nitrogen atom with a one pair of electrons capable of reacting with an acid such that the nitrogen atom is protonated.

The term “amino” is represented by the structure —NR¹R², where R¹ and R² are independently hydrogen, an alkyl group, an aryl group, or a cycloalkyl group.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “nitrogen-bearing heteroaryl group” as used herein refers to an aromatic group that has at least one nitrogen atom incorporated within the ring of the aromatic group. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, imidazolyl, pyrimidinyl, tetrazolyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of nitrogen-bearing heteroaryl groups include, but are not limited to, pyridinyl, pyridazinyl, pyrimidinyl, pyrazinyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl. Fused aryl groups including, but not limited to, indene and naphthalene groups are also contemplated.

The term “secondary amide” is represented by the structure —C(O)NR¹R², where R¹ and R² are independently hydrogen, an alkyl group, an aryl group, or a cycloalkyl group.

The term “hydrazine group” is represented by the structure (R¹)₂N—N(R²)₂, where R¹ and R² are independently hydrogen, an alkyl group, an aryl group, or a cycloalkyl group. Alternatively, the hydrazine group can be included in a ring such as, for example, a cycloalkyl ring.

The term “guanidinyl group” is represented by the structure —N(R¹)—C(═N)—N(R²)₂, where R¹ and R² are independently hydrogen, an alkyl group, an aryl group, or a cycloalkyl group.

Unless otherwise specified, temperatures referred to herein are based on atmospheric pressure (i.e. one atmosphere).

### Methods for Producing Salts of Organic Molecules

Described herein are methods for producing a salt of a solid organic compound. The method involves (a) combining in the solid state the solid organic compound comprising one or more protonatable groups and an acid composition, wherein the amount of acid in the acid composition is about stoichiometric or less than stoichiometric relative to the one or more protonatable groups of the solid organic compound to produce a precursor mixture; and (b) mixing the precursor mixture by mechanochemical milling to produce the salt of the solid organic compound.

The first step involves selecting and combining in the solid state the solid organic compound and an acid composition. The solid organic molecule is any molecule that possess one or more protonatable groups, which is also referred to as a free base. Examples of such groups include, but are not limited to, an amino group, a nitrogen-bearing heteroaryl group, a secondary amido group, a hydrazine group, or a guanidinyl group. In one aspect, the organic molecule is a pharmaceutical, a nutraceutical, an agrochemical, or an explosive. In another aspect, the organic molecule is an amino acid, a peptide, a protein, a nucleic acid, or a small molecule. In another aspect, the organic molecule is nicotinamide, isonicotinamide, picolinamide, nicotinic acid, isonicotinic acid, picolinic acid, caffeine, theophylline, theobromine, acetaminophen, creatine, metformin, L-lysine, L-histidine, L-alanine, sulfanilamide, isoniazid, amoxicillin, nicorandil, amlodipine, sulfonamide, or pyrazinamide.

In one aspect, the acid used in the methods described herein can be an inorganic acid analog. Examples of inorganic acid analogs include, but are not limited to, a sulfonic acid (e.g., triflic acid, methanesulfonic acid, benezesulfonic acid, etc.), a phosphonic acid, a boronic acid, or any combination thereof. In another aspect, the acid can be an inorganic acid such as, for example, H₂SO₄, HCl, HBr, HI, HClO₄, HNO₃, H₃PO₄, H₃PO₃, HF, HCN, H₂CrO₄, or any combination thereof.

The methods described herein do not use excessive amounts of acid to produce the salts, which prevent the formation of undesirable side products. In one aspect, the amount of acid in the acid composition is about stoichiometric or less than stoichiometric relative to each protonatable group of the solid organic compound. In one aspect, the molar ratio of the acid to stoichiometric or less than stoichiometric relative to each protonatable group of the solid organic compound is from 1:1 to 1:0.1, 1:1 to 1:0.2, 1:1 to 1:0.3, 1:1 to 1:0.4, 1:1 to 1:0.5, 1:1 to 1:0.6, 1:1 to 1:0.7, 1:1 to 1:0.8, or 1:1 to 1:0.9.

In certain aspects, when the solid organic compound includes two or more protonatable groups, it may be desirable to protonate only one protonatable group. In one aspect, when the solid organic compound possesses two or more protonatable groups, the amount of acid in the acid composition is about stoichiometric relative to a single protonatable group of the solid organic compound. For example, nicotinamide possesses two protonatable groups. When, 1:0.5 and 1:1 ratios of free base nicotinamide (Nic) to acid are used, NicH-Nic:H₂O and NicH are produced, respectively, as the protonation of the pyridyl nitrogen of nicotinamide is favorable.

The concentration of the acid in the acid composition can vary depending upon the protonatable group present in the solid organic compound. In one aspect, the concentration of the acid in the acid composition has a concentration of less than 15 M. In another aspect, the concentration of the acid in the acid composition has a concentration of 0.5 M, 1 M, 2 M, 3 M, 4 M, 5M, 6 M, 7M, 8 M, 9M, 10 M, 11 M, 12 M, 13 M, 14 M, or less than 15 M, where any value can be a lower and upper endpoint of a range (e.g., 5 M to 13 M, etc.).

The methods described herein use acid compositions that have very little solvent (e.g., microliter scale relative to the amount of the organic molecule). Since so little solvent is used, the methods described herein are suitable for large scale manufacturing. In one aspect, the acid composition is less than about 10 μL per mg of the solid organic compound to produce a precursor mixture. In another aspect, the acid composition is from 0.1 μL per mg of the solid organic compound to less than about 10 μL per mg of the solid organic compound, or 0.1 μL, 0.5 μL, 1.0 μL, 1.5 μL, 2.0 μL, 2.5 μL, 3.0 μL, 3.5 μL, 4.0 μL, 4.5 μL, 5.0 μL, 5.5 μL, 6.0 μL, 6.5 μL, 7.0 μL, 7.5 μL, 8.0 μL, 8.5 μL, 9.0 μL, or 10.0 μL per mg of the solid organic compound, where any value can be a lower and upper endpoint of a range (e.g., 0.5 μL to 2.0 μL per mg of the solid organic compound).

In one aspect, the acid composition includes an acid and a solvent such as, for example, comprises water, an alcohol (e.g., methanol, ethanol, etc.), or a combination thereof. In another aspect, the acid composition consists of an acid and a solvent such as, for example, comprises water, an alcohol (e.g., methanol, ethanol, etc.), or a combination thereof.

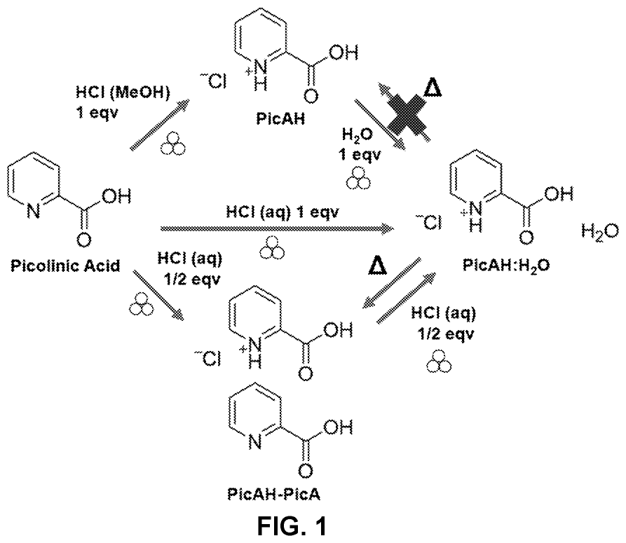

Depending upon the solvent used to produce the acid composition, the salt of the solid organic compound can be an anhydrous compound or a monohydrate compound. For example, if a 1:1 molar ratio of HCl in water and picolinic acid (PicA), caffeine (Caff), and acetaminophen (Acet) are selected, the hydrates picolinic acid HCl monohydrate (PicAH:H₂O), caffeine HCl monohydrate (CaffH:H₂O), and acetaminophen HCl monohydrate (AcetH:H₂O) are formed (FIG. 1). Alternatively, if similar reactions are conducted with HCl (MeOH) or HCl (EtOH), the anhydrous salts picolinic acid HCl (PicAH), caffeine HCl (CaffH), and acetaminophen hemihydrochloride (AcetH-Acet) are formed (FIG. 1). While synthesizing a simple salt is relatively straightforward, forming a hemihydrochloride salt can be more challenging. The methods described herein permit the synthesis of hemihydrate salts.

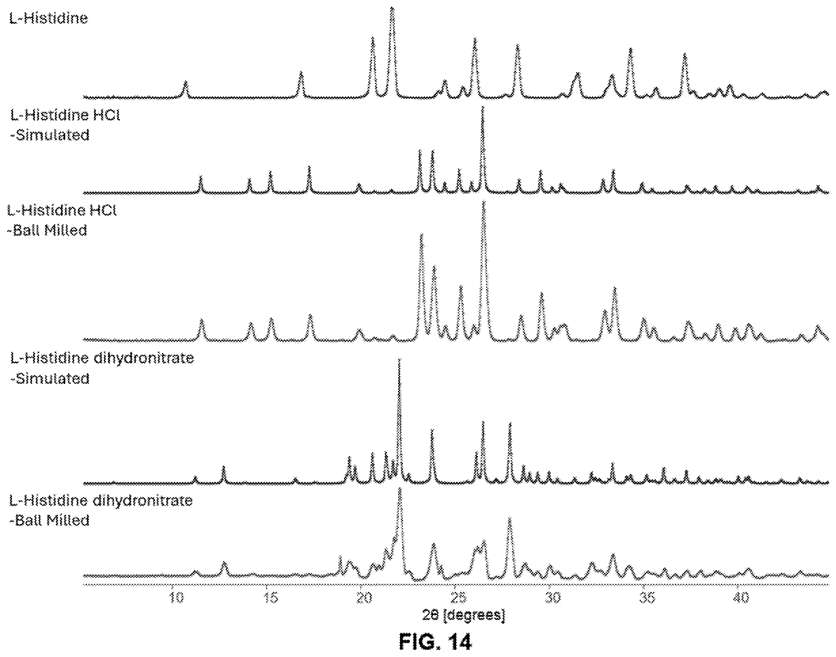

In one aspect, the acid composition can include two or more two or more different acids to produce polyheterosalts or mixed salts, which are generally defined as chemically homogeneous salts characterized by the presence of multiple distinct ions within a single, well-defined stoichiometric compound. For example, a specific mixed salt like L-lysinium L-lysine dichloride nitrate (LysH-Lys-Cl₂/NO₃) or L-lysinium chloride nitrate (LysH-Cl/NO₃) in high purity (e.g., FIG. 14) by altering the amount of each acid used using the methods described herein. Mixed salts can be relevant in pharmaceuticals where the presence of two distinct anions (e.g., Cl⁻ and NO₃⁻, Cl⁻ and Br⁻, etc.) may favorably modify the physicochemical properties (e.g., solubility, bioavailability, stability, etc.) of the solid form of the API. Furthermore, mixed salts of organic compounds can also exhibit unique electrochemical or optical properties, which could be of great value in the design of novel materials with advanced functionalities.

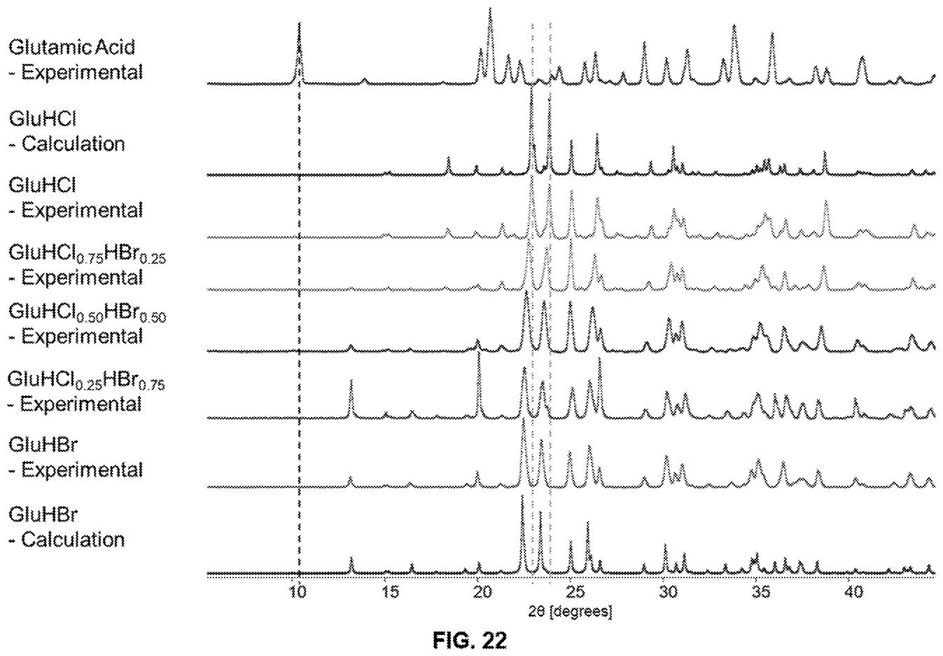

In one aspect, the molar ratio of the sum of the two or more acids is about stoichiometric relative to each protonatable group of the solid organic compound. For example, if the acid composition includes HCl and HBr, the amount of HCl and HBr can be varies such that the molar sum of acid is stoichiometric equivalent to each protonatable group (e.g., 50% HCl/50% HBr, 25% HCl/75% HBr, 75% % HCl/25% HBr, etc.). The Examples provide non-limiting procedures for making polyheterosalts, with an example depicted in FIG. 22.

The solid organic compound and the acid composition are mixed by mechanochemical milling. Mechanochemical milling involves techniques that use mechanical force to drive chemical reactions, typically without the need for solvents or heat. These methods rely on mechanical energy to induce chemical reactions, often leading to faster reaction rates, unique products, and more environmentally friendly processes compared to traditional solution-based chemistry. Examples of such techniques include, but are not limited to, ball milling, resonant acoustic mixing, speed mixing, extrusion, planetary milling, or grinding via mortar and pestle or automated mills.

When conducting mechanochemical milling, the duration and frequency of the milling (i.e., mixing) can be varied. Generally, a sufficient duration is necessary to allow the reaction products to reach an equilibrium state, and higher milling/mixing frequencies speed up the reaction. The milling/mixing frequency can be modified to avoid temperature increases that could adversely affect the reaction between the acid and solid organic molecule. In addition to milling/mixing frequency, the material and volume that is mixed/milled and the nature and size of ball bearings can be modified to control the temperature during mechanochemical milling.

In one aspect, a precursor mixture composed of the solid organic compound and the acid composition is mixed by mechanochemical milling for about 1 minute to about 30 minutes, or 1 minute, 3 minutes, 6 minutes, 9 minutes, 12 minutes, 15 minutes, 18 minutes, 21 minutes, 24 minutes, 27 minutes, or 30 minutes, where any value can be a lower and upper endpoint of a range (e.g., 9 minutes to 15 minutes).

In one aspect, a precursor mixture composed of the solid organic compound and the acid composition is mixed by mechanochemical milling at a temperature of about 20° C. to about 25° C., 20° C., 21° C., 22° C., 23° C., 24° C., or 25° C., where any value can be a lower and upper endpoint of a range (e.g., 21° C. to 23° C.).

In one aspect, a precursor mixture composed of the solid organic compound and the acid composition is mixed by mechanochemical milling at a frequency of about 20 Hz to about 50 Hz, or 20 Hz, 25 Hz, 30 Hz, 35 Hz, 40 Hz, 45 Hz, or 50 Hz, where any value can be a lower and upper endpoint of a range (e.g., 25 Hz to 35 Hz).

In another aspect, mechanochemical milling is performed by liquid assisted grinding (LAG). LAG is mechanochemical method that involves adding a small amount of liquid during grinding or ball milling. The liquid acts as a catalytic medium, enhancing the efficiency of energy transfer between particles without fully dissolving the reactants. The liquid may also be incorporated into the product by forming a solvate. In one aspect, when the acid composition includes a solvent such as water or an alcohol, the solvent can be used to assist the mechanochemical milling of the solid organic compound and the acid of the acid composition.

The methods described herein possess several advantages when compared to compared to conventional techniques for making salts. The methods described herein use at most stoichiometric amounts of acid, often using the most concentrated reagent possible, which greatly reduces both solvent waste and cost. Moreover, using only stoichiometric amounts of the acid is far safer than solvent-based reactions, since dissolution is not required (microliters vs. milliliters on lab scale translates to milliliters vs. liters on a larger scale). For example, to produce organic HCl salts, the methods described herein do not require the material to be dissolved in any solvent.

In many cases, slow evaporation and recrystallization can take anywhere from days to months to produce the salts. The methods described herein permit the production of organic salts (microcrystalline to amorphous) in between 1-30 minutes. Undesired side reactions are less likely to occur using the methods described herein, as there is no excess of acid facilitating acid hydrolysis reactions and/or polymorphic crystallization due to spatial heterogeneity (i.e., temperature or concentration gradients).

Finally, since so little solvent is used, the methods described herein are eminently suitable for large scale manufacturing. At an industrial scale, the methods described herein represent a great potential for monetary savings and increased efficiency, benefiting both the manufacturer and consumer, since it requires very little solvent and energy input (i.e., no heating), reduces solvent waste and impurities, and nets almost quantitative yields (i.e., atom economy). The methods can also be employed for the “on-demand” production of pharmaceuticals that maybe unstable, allowing for in-house investigations of new solid forms and the capability to rapidly produce dosage forms where the API is stabilized. This is particularly advantageous when another property, such as exceptional bioavailability, makes the salt highly desirable despite its limited stability.

### Aspects

Aspect 1. A method for producing a salt of a solid organic compound, the method comprising:
combining in the solid state the solid organic compound comprising one or more protonatable groups and an acid composition, wherein the amount of acid in the acid composition is about stoichiometric or less than stoichiometric relative to the one or more protonatable groups of the solid organic compound to produce a precursor mixture; and
mixing the precursor mixture by mechanochemical milling to produce the salt of the solid organic compound.
Aspect 2. The method of Aspect 1, wherein the acid comprises an inorganic acid analog.
Aspect 3. The method of Aspect 2, wherein the inorganic acid analog comprises a sulfonic acid, a phosphonic acid, a boronic acid, or any combination thereof.
Aspect 4. The method of Aspect 1, wherein the acid comprises an inorganic acid.
Aspect 5. The method of Aspect 4, wherein the inorganic acid comprises H₂SO₄, HCl, HBr, HI, HClO₄, HNO₃, H₃PO₄, H₃PO₃, HF, HCN, H₂CrO₄, or any combination thereof.
Aspect 6. The method of any one of Aspects 1-5, wherein the acid composition is less than about 10 μL per mg of the solid organic compound.
Aspect 7. The method of any one of Aspects 1-5, wherein the acid composition is from 0.1 μL per mg of the solid organic compound to about 10 μL per mg.
Aspect 8. The method of any one of Aspects 1-7, wherein the acid composition comprises water, an alcohol, or a combination thereof.
Aspect 9. The method of any one of Aspects 1-7, wherein the acid composition consists of water, an alcohol, or a combination thereof.
Aspect 10. The method of any one of Aspects 1-9, wherein the acid composition has a concentration of less than 15 M.
Aspect 11. The method of any one of Aspects 1-9, wherein the acid composition has a concentration of 0.5 M to less than 15 M.
Aspect 12. The method of any one of Aspects 1-11, wherein the mechanochemical milling comprises ball milling, resonant acoustic mixing, speed mixing, extrusion, planetary milling, or grinding via mortar and pestle or an automated mill.
Aspect 13. The method of any one of Aspects 1-12, wherein the precursor mixture is mixed by mechanochemical milling for about 1 minute to about 30 minutes.
Aspect 14. The method of any one of Aspects 1-13, wherein the precursor mixture is mixed by mechanochemical milling at a temperature of about 20° C. to about 25° C.
Aspect 15. The method of any one of Aspects 1-14, wherein the precursor mixture is mixed by mechanochemical milling at a frequency of about 20 Hz to about 50 Hz.
Aspect 16. The method of any one of Aspects 1-15, wherein the protonatable group comprises a nitrogen bearing group.
Aspect 17. The method of any one of Aspects 1-16, wherein the protonatable group comprises an amino group, a nitrogen-bearing heteroaryl group, a secondary amido group, a hydrazine group, or a guanidinyl group.
Aspect 18. The method of any one of Aspects 1-17, wherein the solid organic compound comprises a pharmaceutical, a nutraceutical, an agrochemical, or an explosive.
Aspect 19. The method of any one of Aspects 1-17, wherein the solid organic compound comprises an amino acid, a peptide, a protein, a nucleic acid, or a small molecule.
Aspect 20. The method of any one of Aspects 1-17, wherein the solid organic compound comprises nicotinamide, isonicotinamide, picolinamide, nicotinic acid, isonicotinic acid, picolinic acid, caffeine, theophylline, theobromine, acetaminophen, creatine, metformin, L-lysine, L-histidine, L-alanine, sulfanilamide, isoniazid, amoxicillin, nicorandil, amlodipine, sulfonamide, or pyrazinamide.
Aspect 21. The method of any one of Aspects 1-20, wherein the acid composition comprises two or more different acids.
Aspect 22. The method of any one of Aspects 1-20, wherein the amount of acid in the acid composition is about stoichiometric relative to each protonatable group of the solid organic compound.
Aspect 23. The method of any one of Aspects 1-20, wherein when the solid organic compound possesses two or more protonatable groups, the amount of acid in the acid composition is about stoichiometric relative to a single protonatable group of the solid organic compound.
Aspect 24. The method of any one of Aspects 1-23, wherein the salt of the solid organic compound is an anhydrous compound.
Aspect 25. The method of any one of Aspects 1-23, wherein the salt of the solid organic compound is a monohydrate compound.

### EXAMPLES

### Characterization of Products of Mechanochemical Salification Methods.

### Primary Characterization—Powder X-Ray Diffraction (PXRD)

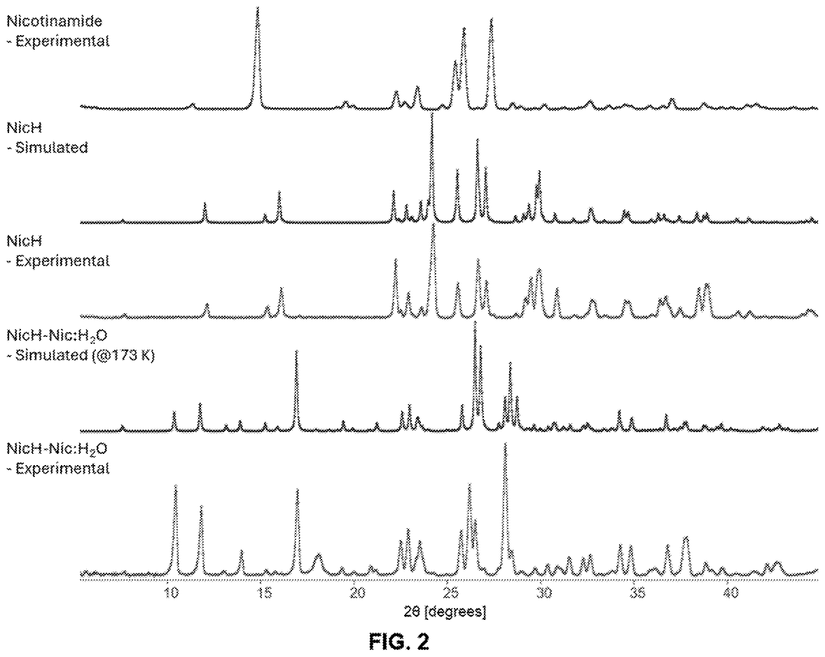

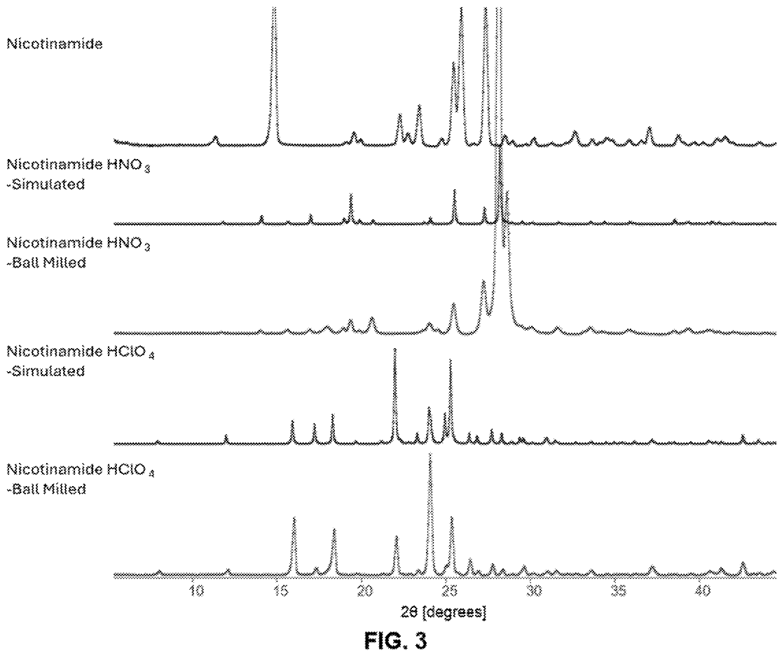

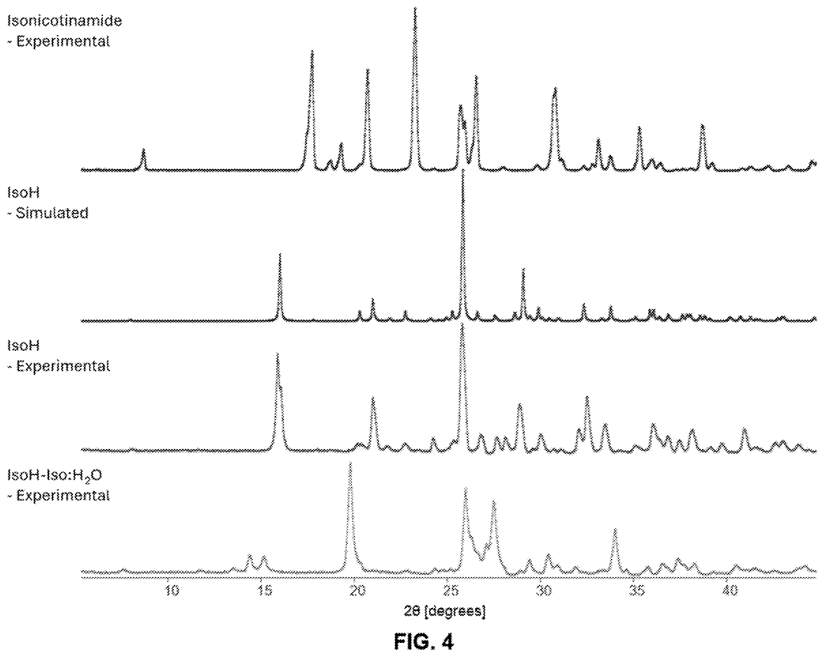

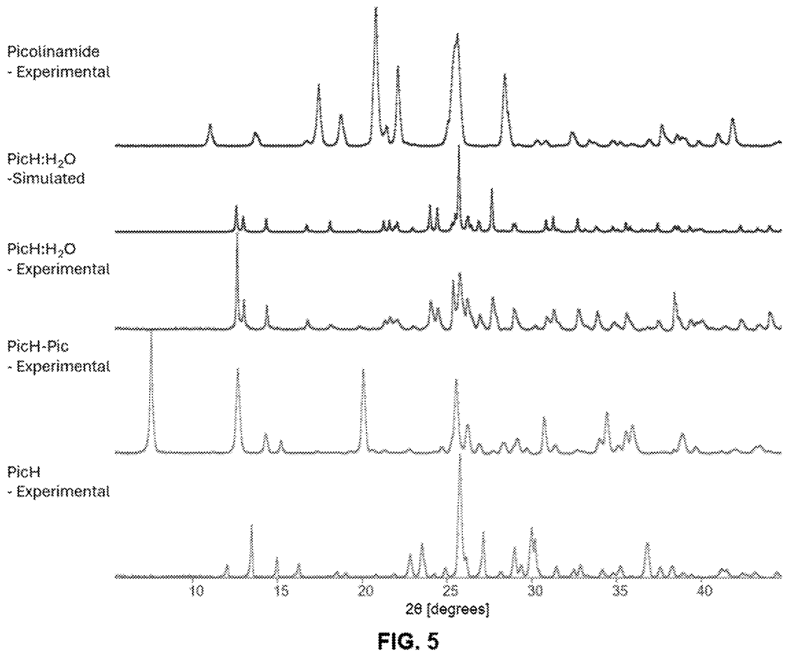

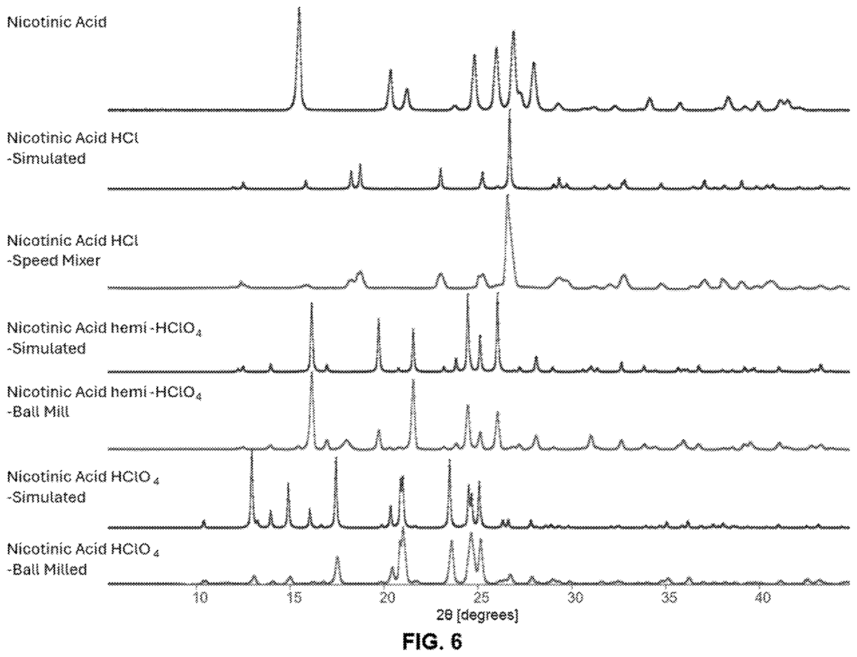

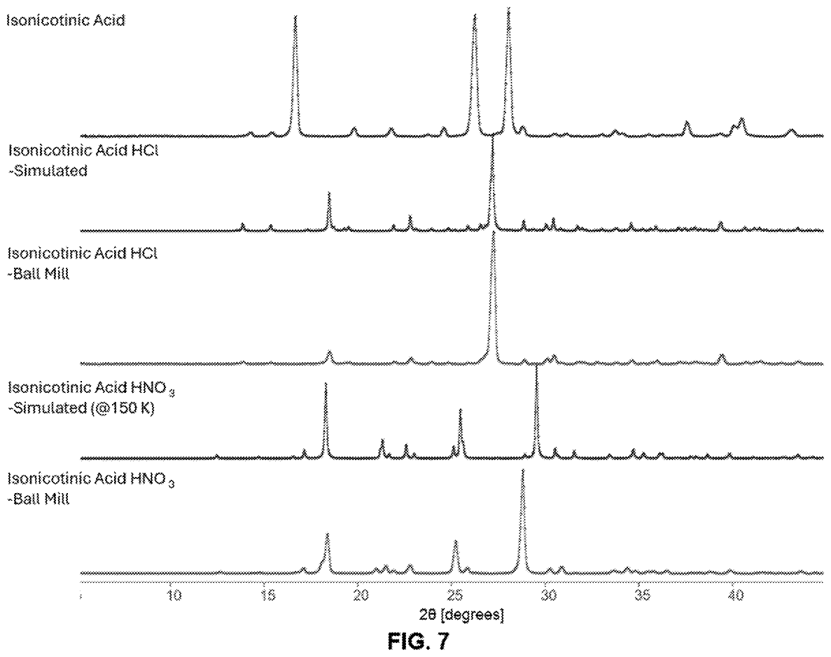

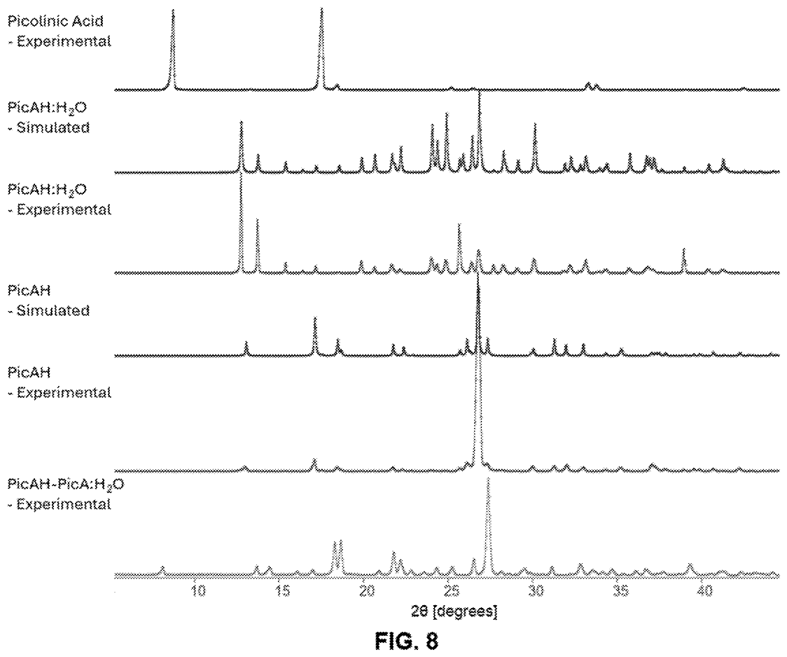

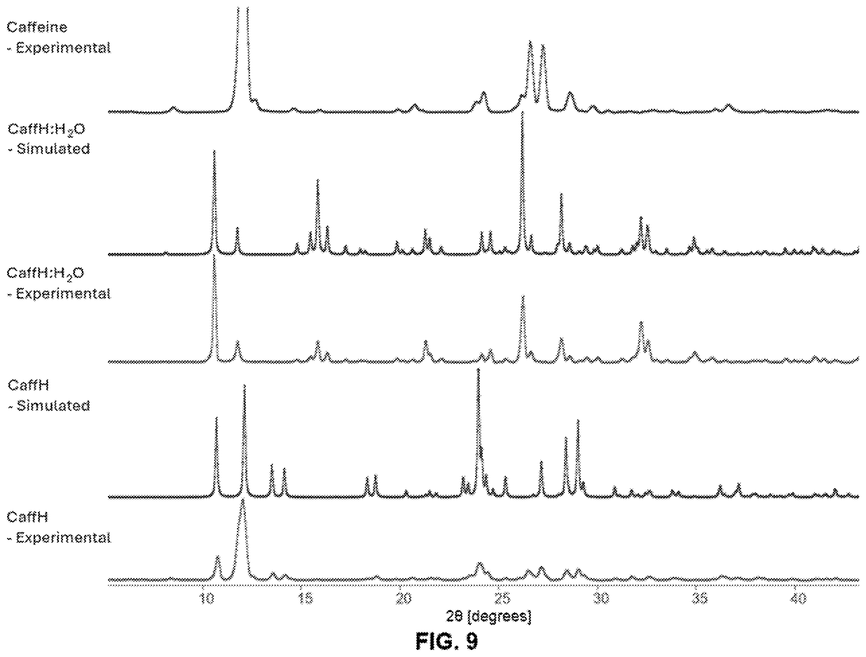

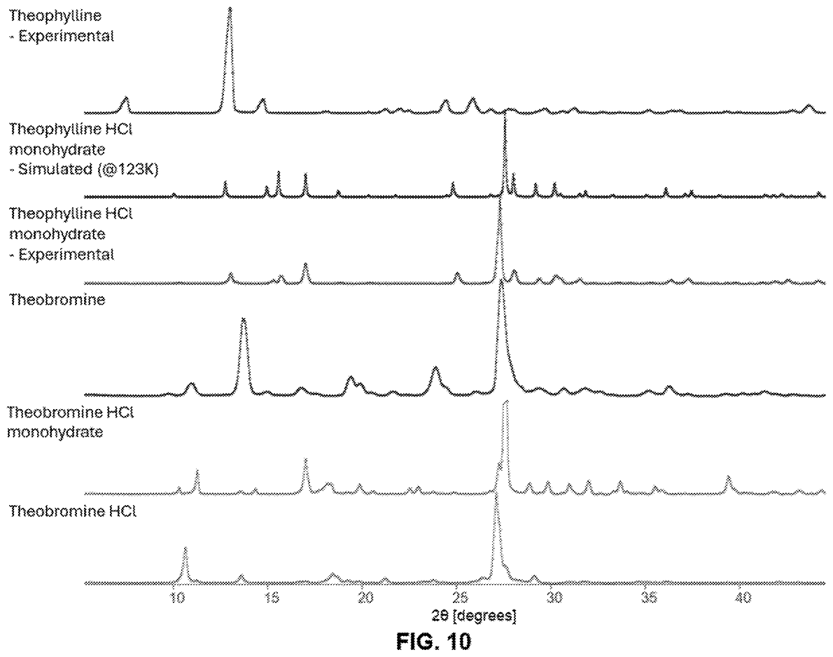

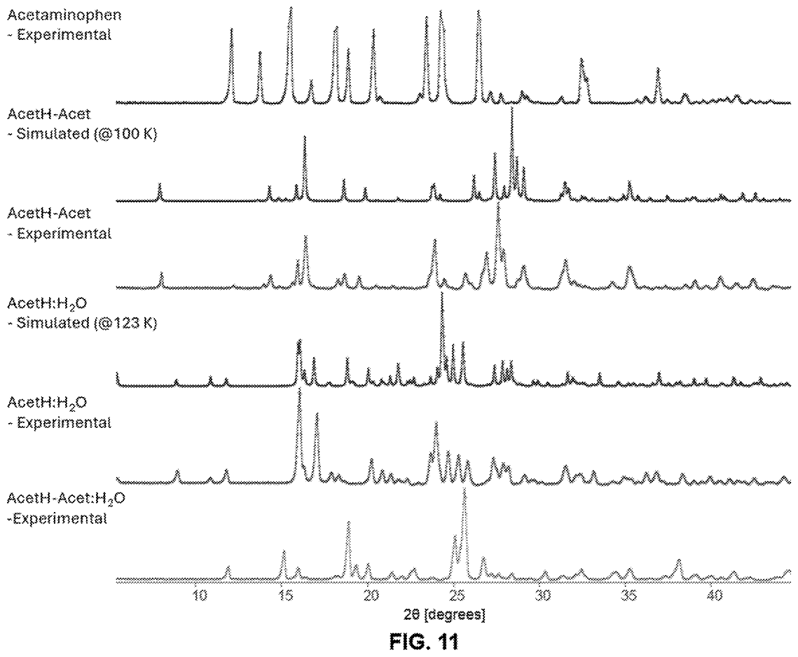

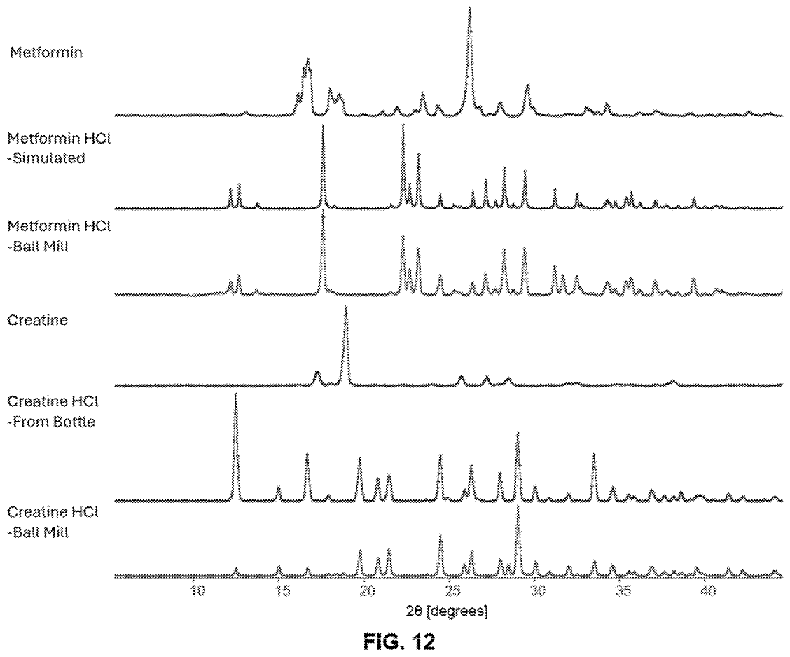

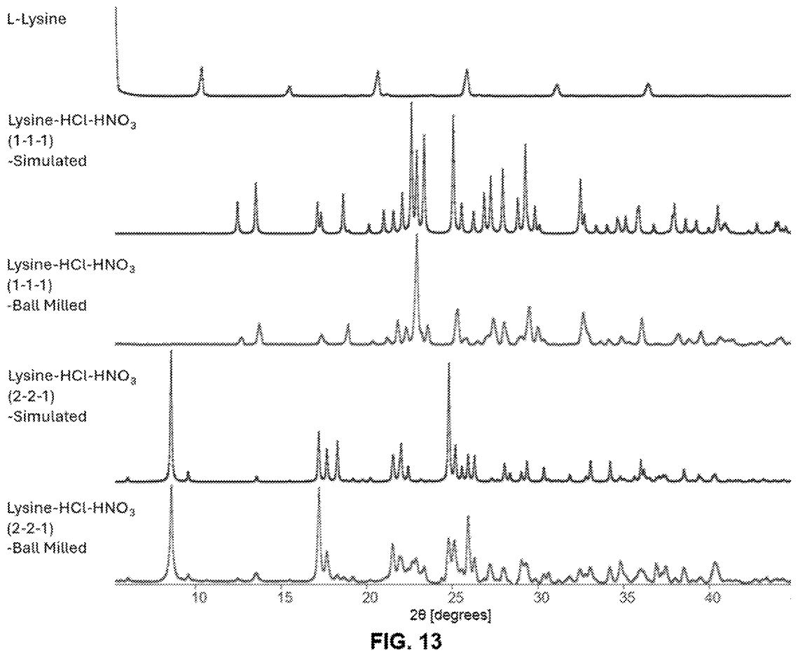

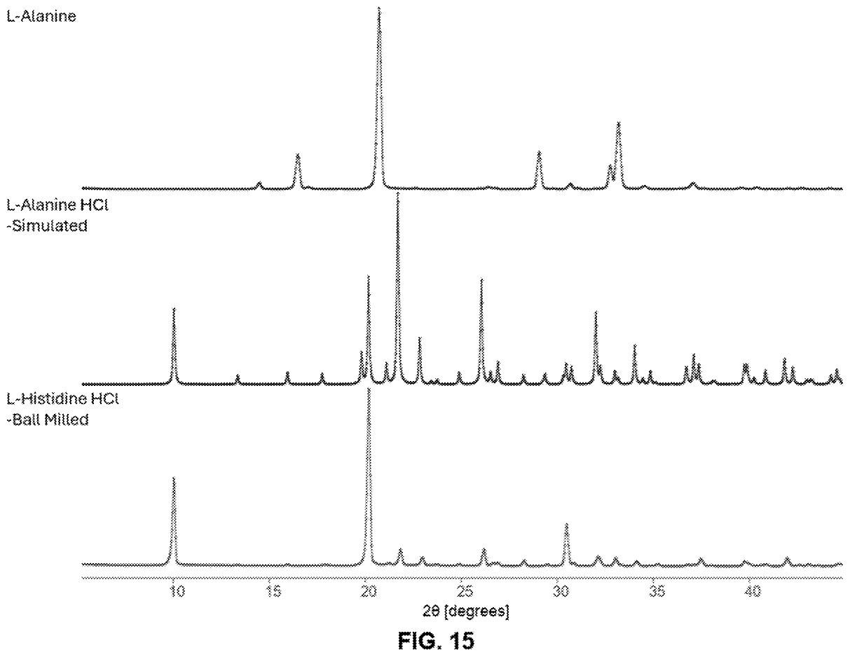

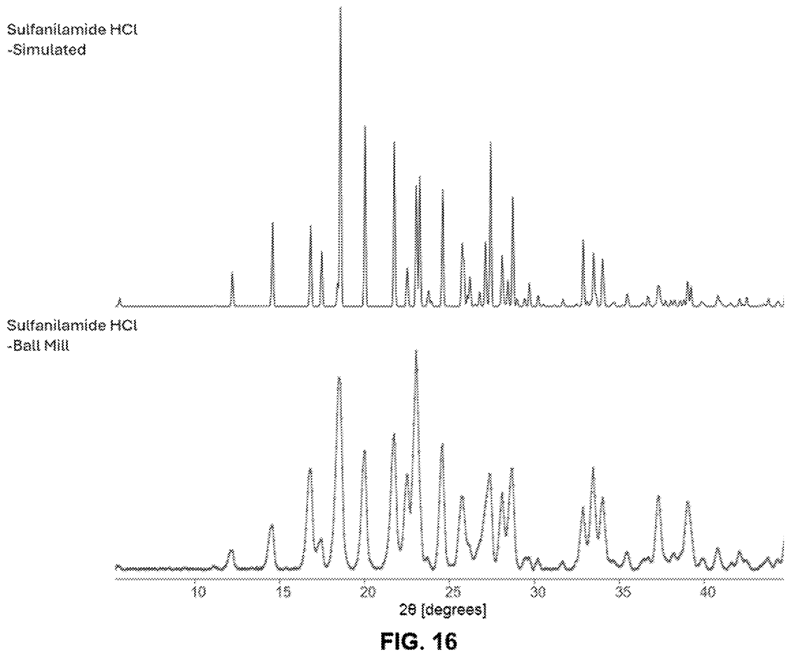

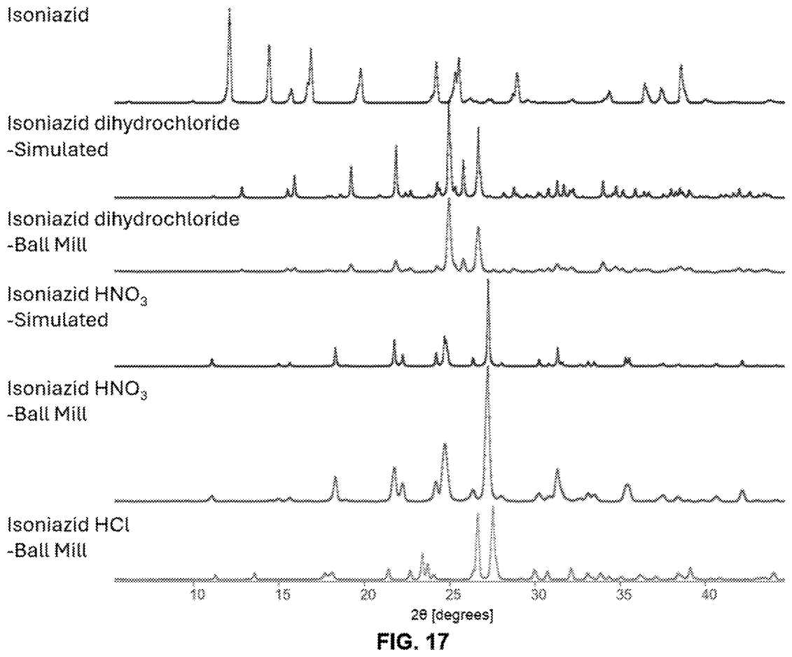

PXRD is a reliable and rapid means of confirming the production of known organic salts. For the samples discussed herein, all of the PXRD patterns of previously known organic salts are in good agreement with simulated patterns based on reported crystal structures (FIGS. 1-16). PXRD patterns of novel salts without known crystal structures are compared to patterns of educts and known salts (FIG. 17). PXRD patterns are also used to confirm the absence of impurities and/or by-products, and are used to confirm the phase identities of single crystals and powdered microcrystalline samples.

### Secondary Characterization—³⁵Cl Solid-State NMR (SSNMR)

Solid-state ¹³C SSNMR spectroscopy is widely used to characterize organic molecules, APIs, and their salt and cocrystalline forms; however, in many instances, it is not always useful for unambiguously differentiating polymorphs and solvates of different solid forms, due to subtle differences in ¹³C chemical shifts that are not always easy to interpret.

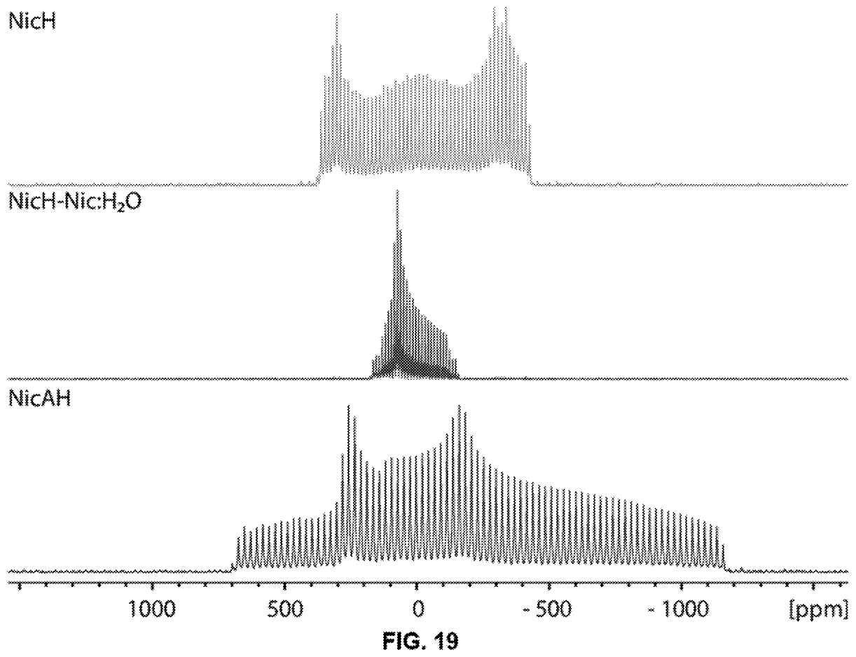

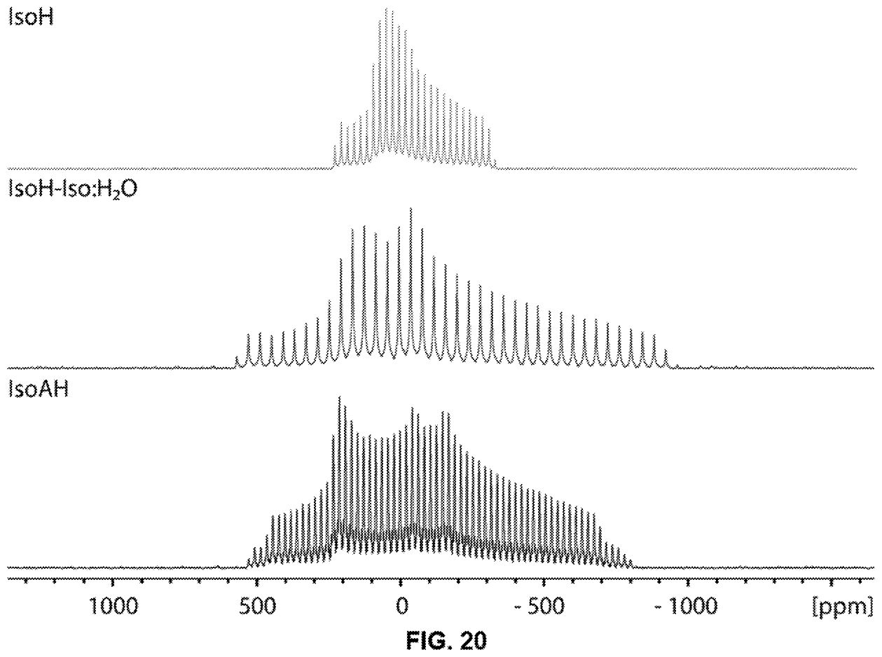

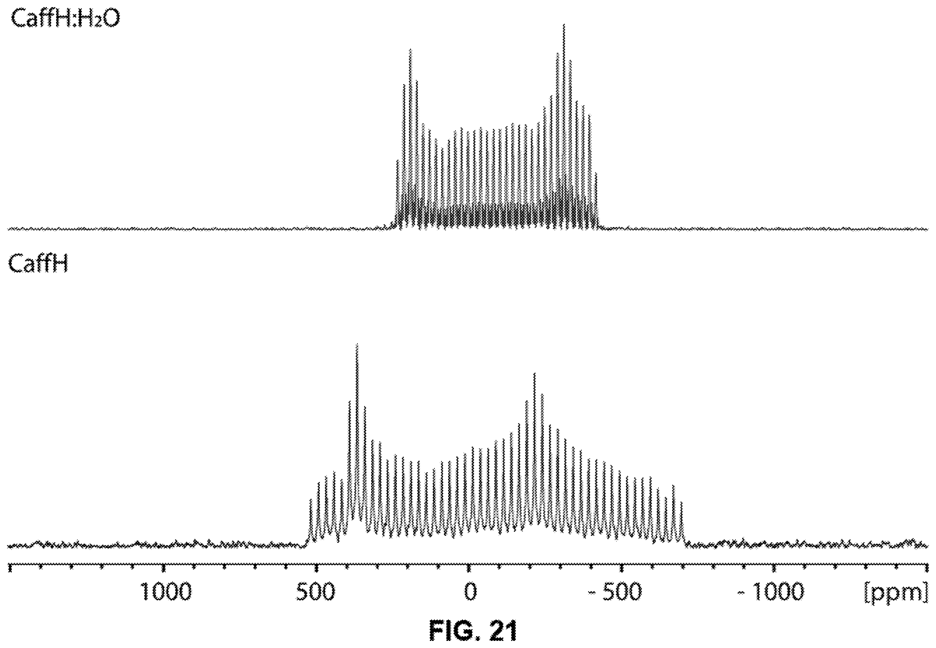

³⁵Cl SSNMR was used for this purpose, since ³⁵Cl SSNMR powder patterns for each solid form help distinguish the unique chloride ion environments. This is because ³⁵Cl SSNMR spectra are primarily influenced by quadrupolar interactions, which arise from the electric field gradients (EFGs) centered at the origin of each ³⁵Cl nucleus. These EFGs are highly sensitive to the local hydrogen bonding environments surrounding the chloride ions, thereby yielding a unique ³⁵Cl SSNMR spectral fingerprint (cf. FIGS. 19-21). Furthermore, ³⁵Cl SSNMR is extremely useful for studying the relationships between the ³⁵Cl EFGs and hydrogen bonding environments, easily distinguishing the number of hydrogen bonds at a chloride ion site, and providing a robust probe of ionic vs. covalent bonding.

Table 1 provides salts of organic molecules that were produced by the methods described herein.

TABLE 1
Solid forms of various molecules formed with our method.

| | | Corresponding Figures | |
|---|---|---|---|
| Molecule | Solid Forms | PXRD | ³⁵CI SSNMR^{†} |
| Nicotinamide | Nicotinamide HCl | 2, 3 | 19 |
| | Nicotinamide hemihydrochloride monohydrate | | |
| | Nicotinamide HNO₃ | | |
| | Nicotinamide HClO₄ | | |
| Isonicotinamide | Isonicotinamide HCl | 4 | 20 |
| | Isonicotinamide hemihydrochloride monohydrate* | | |
| Picolinamide | Picolinamide HCl* | 5 | n/a |
| | Picolinamide HCl monohydrate* | | |
| | Picolinamide hemihydrochloride* | | |
| Nicotinic Acid | Nicotinic Acid HCl | 6 | 19 |
| | Nicotinic Acid HClO₄ | | |
| | Nicotinic Acid hemihydroperchlorate | | |
| Isonicotinic Acid | Isonicotinic Acid HCl | 7 | 20 |
| | Isonicotinic Acid HNO₃ | | |
| Picolinic Acid | Picolinic Acid HCl | 8 | n/a |
| | Picolinic Acid HCl monohydrate | | |
| | Picolinic Acid hemihydrochloride monohydrate* | | |
| Caffeine | Caffeine HCl | 9 | 21 |
| | Caffeine HCl monohydrate | | |
| Theophylline | Theophylline HCl | 10 | n/a |
| Theobromine | Theobromine HCl* | 11 | n/a |
| | Theobromine HCl monohydrate* | | |
| Acetaminophen | Acetaminophen HCl monohydrate | 12 | n/a |
| | Acetaminophen hemihydrochloride | | |
| | Acetaminophen hemihydrochloride monohydrate* | | |
| Creatine | Creatine HCl | 12 | n/a |
| Metformin | Metformin HCl | 13 | n/a |
| L-Lysine | L-Lysinium chloride nitrate | 14 | n/a |
| | L-Lysine L-lysinium dichloride nitrate | | |
| L-Histidine | L-Histidine HCl | 14 | n/a |
| | L-Histidine dihydronitrate | | |
| L-Alanine | L-Alanine HCl | 15 | n/a |
| Sulfanilamide | Sulfanilamide HCl | 16 | n/a |
| Isoniazid | Isoniazid HCl* | 17 | n/a |
| | Isoniazid dihydrochloride | | |
| | Isoniazid HNO₃ | | |
| Amoxicillin | Amoxicillin HCl* | 18 | n/a |
| Nicorandil | Nicorandil hemihydronitrate* | 19 | n/a |

^{†}Only for select HCl salts.
*Indicates materials that have not been previously reported.

### Preparation of Polyheterosalts

Two or more acids can be combined with the solid organic molecule to produce polyheterosalts or mixed salts, which are generally defined as chemically homogeneous salts characterized by the presence of multiple distinct ions within a single, well-defined stoichiometric compound. For example, ionic solid solutions of L-glutamic acid were prepared using the methods described herein for the simultaneous incorporation of hydrochloric acid and hydrobromic acid in variable stoichiometric ratios. Referring to FIG. 22, PXRD patterns show small but systematic shifts in peak positions while remaining isostructural with the parent GluHCl and GluHBr phases, consistent with formation of mixed-halide ionic solid solutions. Notably, reflections observed in GluHCl at 20=22.9° and 23.9° shift to lower diffraction angles with increasing bromide incorporation, indicating expansion of the unit cell consistent with substitution of chloride by the larger bromide ion. This approach provides a clear synthetic route for controlled incorporation of mixed counterions within the crystal lattice, enabling preparation of ionic solid solutions with tunable stoichiometry and tailored physicochemical properties.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A method for producing a salt of a solid organic compound, the method comprising:
(a) combining in the solid state the solid organic compound comprising one or more protonatable groups and an acid composition, wherein the amount of acid in the acid composition is about stoichiometric or less than stoichiometric relative to the one or more protonatable groups of the solid organic compound to produce a precursor mixture; and
(b) mixing the precursor mixture by mechanochemical milling to produce the salt of the solid organic compound.

2. The method of claim 1, wherein the acid comprises an inorganic acid analog.

3. The method of claim 2, wherein the inorganic acid analog comprises a sulfonic acid, a phosphonic acid, a boronic acid, or any combination thereof.

4. The method of claim 1, wherein the acid comprises an inorganic acid.

5. The method of claim 4, wherein the inorganic acid comprises H₂SO₄, HCl, HBr, HI, HClO₄, HNO₃, H₃PO₄, H₃PO₃, HF, HCN, H₂CrO₄, or any combination thereof.

6. The method of claim 1, wherein the acid composition is less than about 10 μL per mg of the solid organic compound.

7. The method of claim 1, wherein the acid composition consists of water, an alcohol, or a combination thereof.

8. The method of claim 1, wherein the acid composition has a concentration of less than 15 M.

9. The method of claim 1, wherein the mechanochemical milling comprises ball milling, resonant acoustic mixing, speed mixing, extrusion, planetary milling, or grinding via mortar and pestle or an automated mill.

10. The method of claim 1, wherein the precursor mixture is mixed by mechanochemical milling for about 1 minute to about 30 minutes.

11. The method of claim 1, wherein the precursor mixture is mixed by mechanochemical milling at a temperature of about 20° C. to about 25° C.

12. The method of claim 1, wherein the precursor mixture is mixed by mechanochemical milling at a frequency of about 20 Hz to about 50 Hz.

13. The method of claim 1, wherein the protonatable group comprises a nitrogen bearing group.

14. The method of claim 1, wherein the protonatable group comprises an amino group, a nitrogen-bearing heteroaryl group, a secondary amido group, a hydrazine group, or a guanidinyl group.

15. The method of claim 1, wherein the solid organic compound comprises a pharmaceutical, a nutraceutical, an agrochemical, or an explosive.

16. The method of claim 1, wherein the solid organic compound comprises an amino acid, a peptide, a protein, a nucleic acid, or a small molecule.

17. The method of claim 1, wherein the acid composition comprises two or more different acids.

18. The method of claim 1, wherein the amount of acid in the acid composition is about stoichiometric relative to each protonatable group of the solid organic compound.

19. The method of claim 1, wherein when the solid organic compound possesses two or more protonatable groups, the amount of acid in the acid composition is about stoichiometric relative to a single protonatable group of the solid organic compound.

20. The method of claim 1, wherein the salt of the solid organic compound is an anhydrous compound or a monohydrate compound.
